(19) United States
(12) Patent Application Publication
(10) Pub. No.: US 2022/0317434 A1
(43) Pub. Date: Oct. 6, 2022

(54) Micro-organism identification using light microscopes, conveyor belts, static electricity, artificial intelligence and machine learning algorithms

(71) Applicant: Keith Louis De Santo, St Petersburg, FL (US)

(72) Inventor: Keith Louis De Santo, St Petersburg, FL (US)

(21) Appl. No.: 17/221,776

(22) Filed: Apr. 3, 2021

(51) Int. Cl. G02B 21/36; G06N 20/00; C12M 1/12; C12M 1/26

(52) U.S. Cl. CPC G02B 21/367; G06N 20/00; C12M 25/08; C12M 33/08; C12M 33/16; G01N 24/08

(57) ABSTRACT

This new application can be administered completely by artificial Intelligence or manually while utilizing the artificial intelligence platform enabling cost savings. This new artificial intelligence application is a breakthrough in that it is based on highly accurate data which leads to learned behaviors of micro-organisms. This data is applied to accurate management and elimination of threats from micro-organisms to people, plants and animals using biosurfactants. This new cost-efficient AI conveyor belt micro-organism application gets us closer to understanding what lurks around us.

This new application can be administered completely by artificial Intelligence or manually while utilizing the artificial intelligence platform enabling cost savings.

Without accuracy of micro-organism identification, the foundation built upon that data will crumble and the technology, money invested and time spent will only further our understanding of what is next, how to prepare for it and how to eliminate it before it eliminates. Factual data in, factual data evaluated, priceless data out. This new artificial intelligence application is a breakthrough in that it is based on highly accurate factual data, which leads to learned accurate behaviors of micro-organisms in turn leads to accurate management and elimination of threats from micro-organisms to people, plants and animals. This new cost-efficient AI conveyor belt micro-organism application gets us closer to understanding what lurks around us.

The Five Phases of the Artificial Intelligence Micro-Organism Identification Application. “AIMOIA”

Obtaining the micro-organism or Pollutant

Accurate identification of the micro-organism

Learning the behaviors of all micro-organisms

Accurate elimination of threats from micro-organisms.

Deterrent of micro-organisms formation and buildup.

Artificial Intelligence is based on machines (mechanical machines equipped with CPUs) having the ability to perform activities that are normally thought to require intelligence from humans.

Machine learning algorithms are just that, they learn. Artificial Intelligence combined with machine learning leads to faster processing, more accurate data with the ability to avoid mistakes that humans make when performing tasks. Machine learning of micro-organisms will lead to a better understanding of micro-organisms and how to manage the threats they pose.

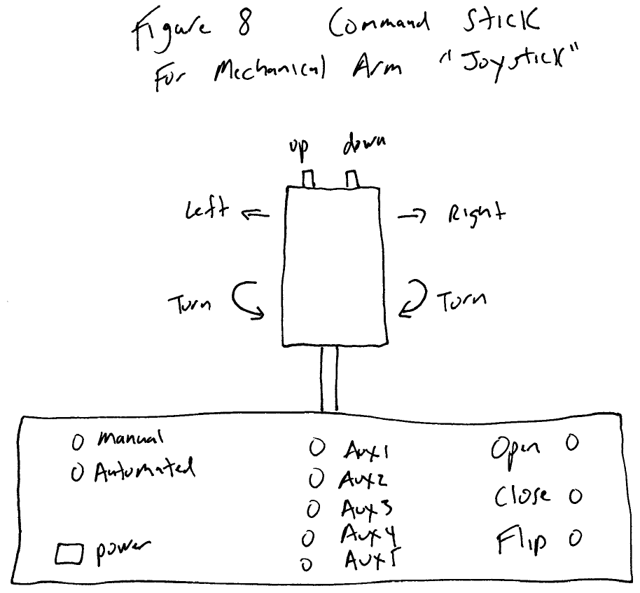

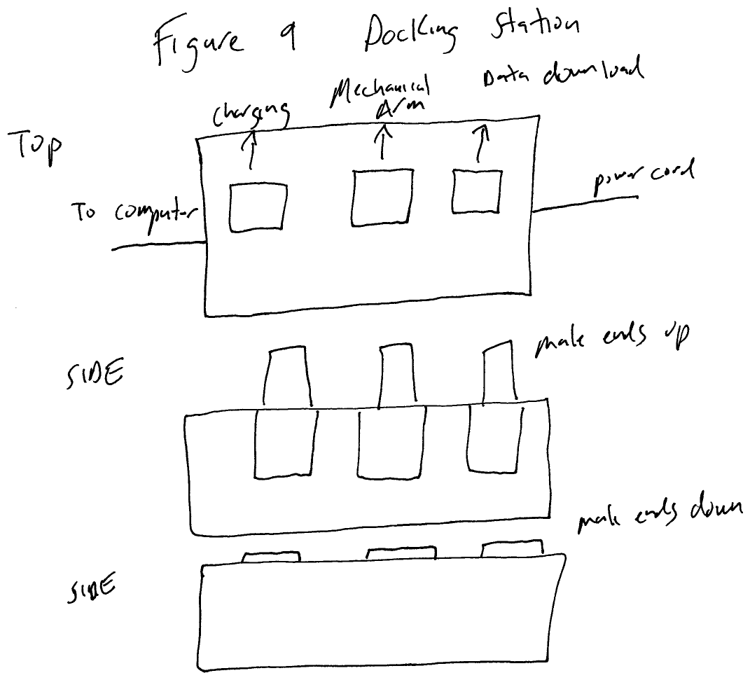

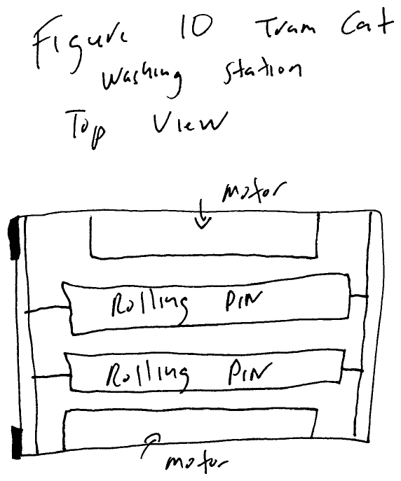

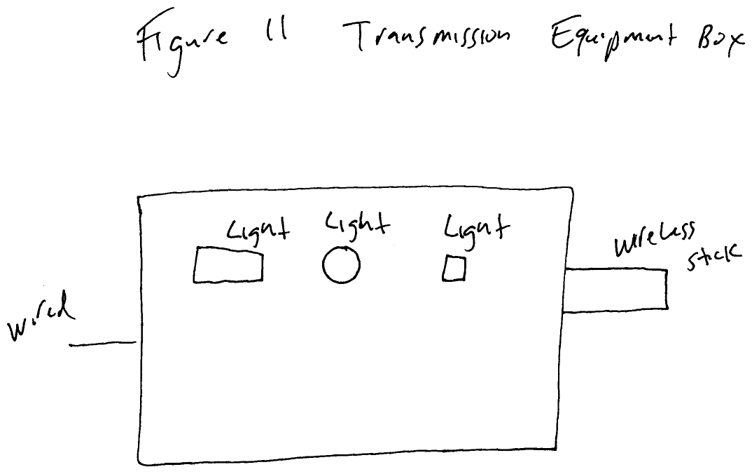

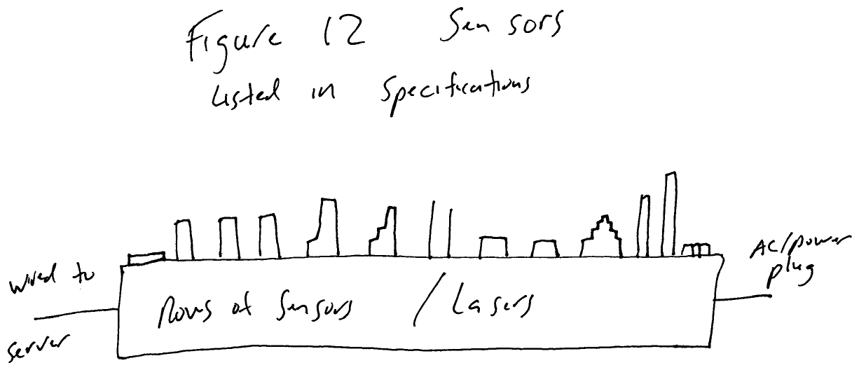

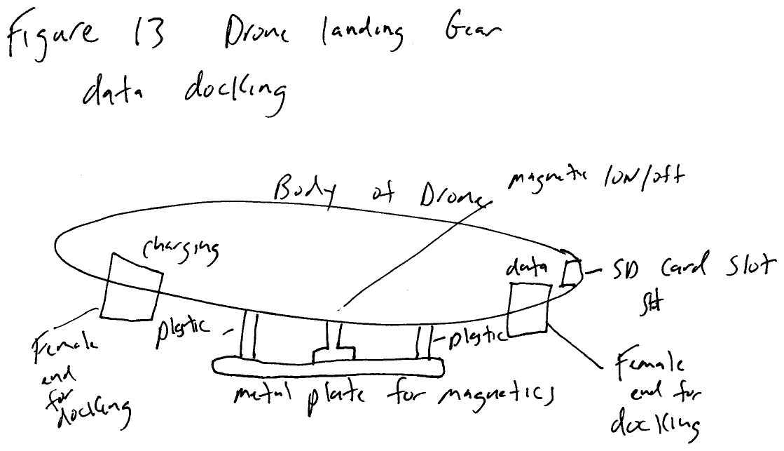

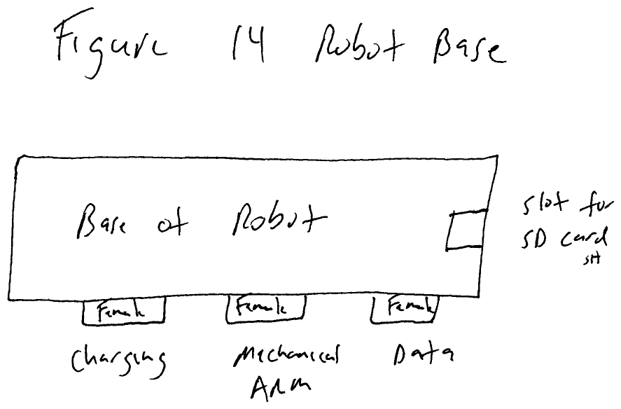

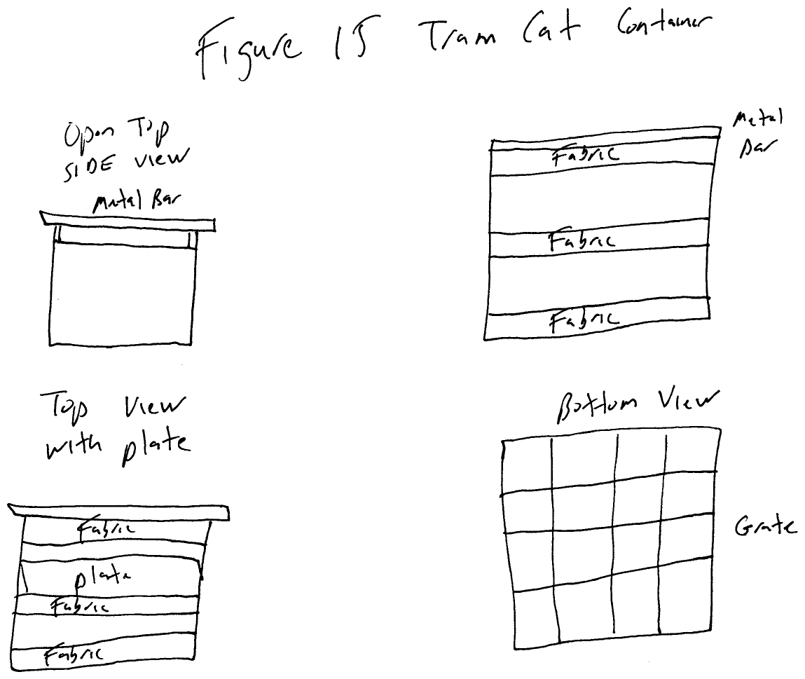

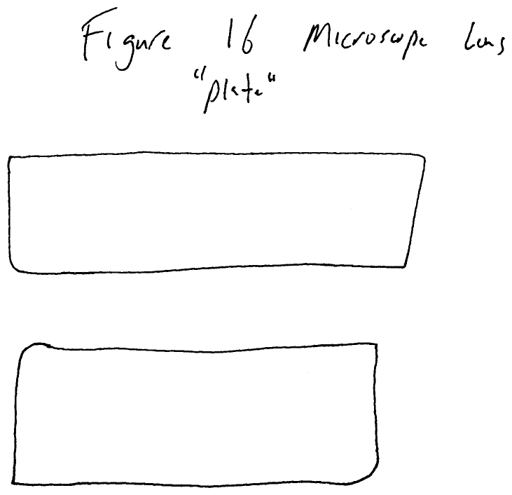

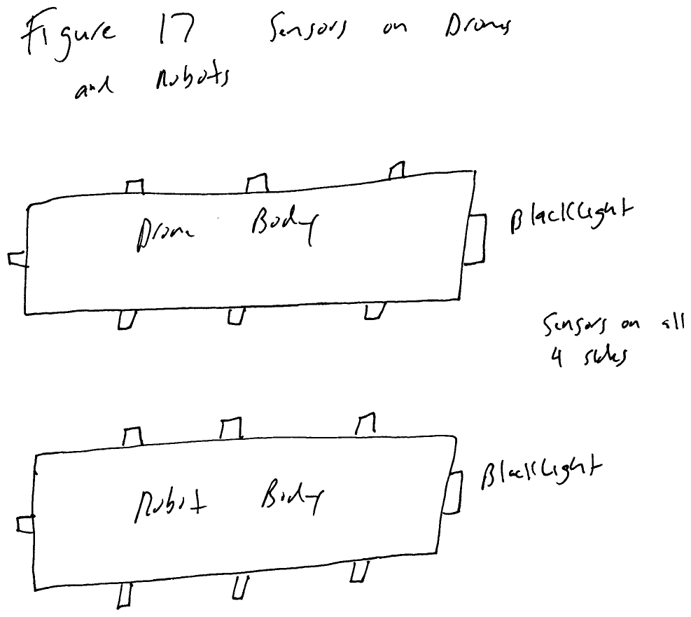

Artificial Intelligence Micro-Organism Identification Application or “AIMOIA” captures Micro-organisms on surfaces and in the air for evaluation and elimination. The new application will be referred hereafter as the AI platform which includes artificial intelligence and machine learning algorithms, an extensive database of micro-organism data which includes pictures, weight, height, length, width, colors, circumference, diameter and ages of singular and clusters of micro-organisms. The hardware consists of drones, robots (with mechanical arms), containers holding glass microscope slides, glass microscope slides with static electricity, conveyor belts, light and electron microscopes, x-ray and NMR machines, wired (FIG. 7) and wireless data transmission equipment, desktop computers and servers. The AI platform identifies airborne and surface micro-organisms, tiny matter, learns, evaluates, identifies trends and forecasts events regarding micro-organisms, their behaviors, their threats and how to identify and eliminate them accurately. This new application can be applied in two separate forms, a manual comprehensive less expensive application or a more expensive application that is completely managed by the AI platform. Both forms are evaluated by the AI platform either locally or from great distances from individual servers, server farms or connected server farms.

Individual components or a combination of components of the new application can be chosen manually or the AI platform will make the choices that are best suited for what is requested. This new application can detect microbes from 10 to the minus 10 microns up to 1 micron in size which includes mold, fungus, allergens, bacteria and viruses in homes and businesses. The application offers an A La Carte menu for micro-organism identification and elimination and which application or combination of applications fit certain budgets and time constraints.

As the world has come to know, micro-organisms and their effects on society have led to loss of life, negative financial impacts and a state of living in fear. This application can identify disease causing micro-organisms, eliminate those threats to humans, plants and animals and deter those threats through biosurfactant biofilm applications.

The Five Phases of the Artificial Intelligence Platform. Obtaining the micro-organism

Accurate identification of the micro-organism Learning the behaviors of all micro-organisms Accurate elimination of threats from micro-organisms. Deterrent of micro-organisms formation and buildup.

Stationary and Mobile

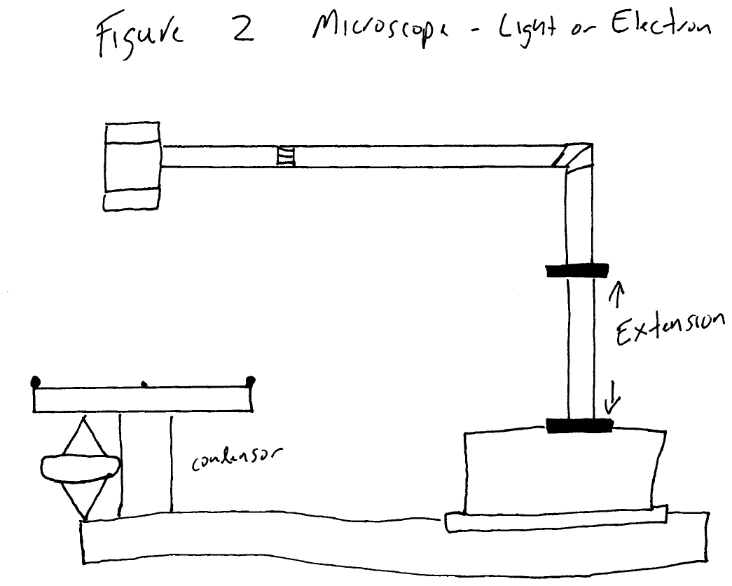

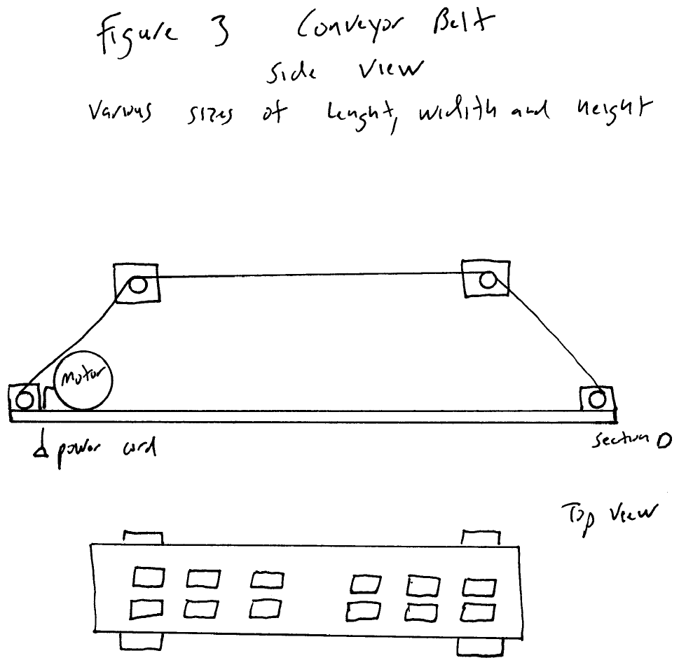

The stationary aspect of the platform basically consists of a light microscope (or electron microscope (FIG. 2), and at certain times an X-ray Artificial Intelligence managed conveyor belt, microscope slides charged with static electricity, a desktop computer, data transmission equipment, artificial intelligence and machine learning algorithms. Other components are listed at the end of this document.

The mobile aspect of the platform consists basically of a drone, a robot with mechanical arm, prop wash application, tire vibration application and TRAnsportable Microorganism CATch ContainerS “TRAM CATS” Other components are available and discussed later.

The elimination of micro-organisms component of the platform is managed by machine learning and artificial intelligence algorithms and their management of biosurfactants.

The drones and robots can be equipped with laser sensors, environmental sensors, high definition cameras, mechanical arms, and transmission equipment. Other components are also available and discussed later.

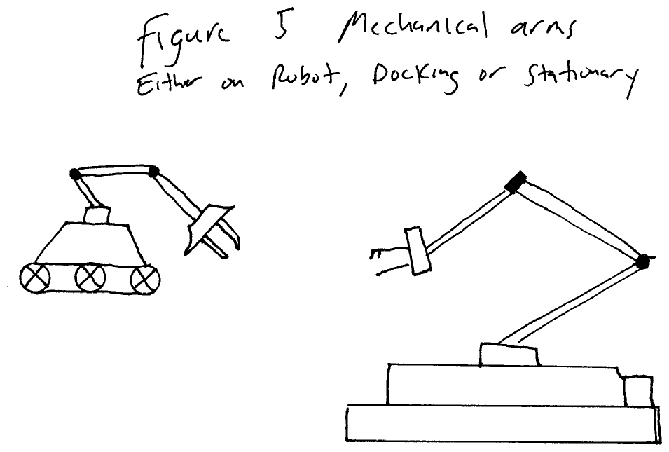

All of the AI platforms hardware components such as drones, robots, mechanical arms, sensors, Tram Cats, transmission data equipment, light microscope or electron microscope, AI conveyor belt, cameras for streaming video and high definition pictures, batteries, microprocessor chips (Computers or CPUs-FIG. 4), can be normal size (contemporary, of this time period), nanotechnology sized or a combination of both.

Artificial Intelligence Micro-Organism Identification Application is just that, an application to identify micro-organisms for accurate elimination for indoor structures. It can run as a single

platform or be connected to several other platforms close by or many miles away or be connected to many server farms. Both stationary and mobile components work in conjunction with each other and can be managed by a touch screen, voice, text or a combination of all three. The application is designed to work from a command center for corporations or can be local for a small residence. Cameras can be mounted on the drones and robots for visual management from a location other than the one being surveyed for micro-organisms.

Select components of the system can be managed manually by an individual or a group of individuals for a basic understanding of their environment or can be completely managed by the AI platform with high accuracy.

The system is plug and go after some basic assembly, plug it in and it is ready for operation. Electricity is needed but the operation can run on a generator powered by either one or a combination of natural gas, gasoline, hydrogen fuel cells or nuclear power. One individual can operate the application up to 5,000 cubic feet but over 5,000 cubic feet, groups of individuals may be needed depending on the layout and the degree of clutter in the space. The AI platform is designed to manage the application with millions of cubic feet being managed at one time.

The application platform was developed for users who desire choices of service, a cheaper cost basis, with and without time constraints. The application can be administered manually in part, or with the full artificial intelligence platform application affording accurate results. The application offers an accurate micro-organism identification report (voice or in electronic form with supporting pictures and video), is completely automated, and the duration of the application can be reduced by adding more AI platforms and components. The more platforms that are initialized, and more components, the less time it takes for micro-organism identification and the more accurate the data is. Each additional platform is offered with additional options for obtaining an electrostatic field on various microscope slides. The machine learning algorithms learned how much time was required from other runs to identify micro-organisms needed for identification by learning from previous application runs in similar and various other industries, by how much hardware was used, what type of hardware was used, how was the hardware utilized and how much clutter was on the floor. Clutter for this application is defined as a mass of various items lying on the floor. Application run is defined as how long each 5,000 cubic feet.

takes to scan with a drone, a robot equipped with an environmental sensor and laser mapping equipment. A robot maintains a laser while the drone uses minors and reflectors to help the robot get a 3-dimensional grid drawing of the area including items within the area. The drone at times will be guided by the AI platform to position itself for exact reflection by shutting itself down after arriving at the exact spot the AI platform is requesting. The drone also takes video and pictures where the AI platform compares the pictures and video taken from the drones to the 3-dimensional grid taken from the robots and forms a basis for where micro-organisms are possibly hiding and where to place the microscope slides with static electricity. Items in an area are defined as furniture, appliances, pictures, lighting, items hanging from ceilings (ceiling fans) and windows. These items also add to clutter but will be referred as secondary clutter. The machine learning algorithms work simultaneously with the artificial intelligence prediction algorithms to calculate projected times of runs for 5,000 cubic feet and increments of 5,000 cubic feet thereafter. The AI platform has also learned that some residences have very few micro-organisms and most of those have been innocuous. Some homes with occupants of single females over the age of 30 years old, no pets and with shoes being left at the door have the least micro-organisms. Most micro-organisms have been found in assisted living facilities.

The application explained in choices.

First choice is manual. “The Swab or Slide Testing Kit” for surface and airborne micro-organisms evaluation.

The first choice that the user has is the simplest, cheapest and the most time consuming. We provide swabs and agar slides to the user. Swabbing (rubbing while turning the swab) on a surface such as a door knob and then swabbing the surface of agar slides (petri dishes) with matter from the door knob can give the user a general idea of what types of micro-organisms are on the surfaces in their homes, businesses, school gym locker rooms, food processing plants or assisted living facilities to name a few. Just setting the uncovered agar plate out in the open (without swabbing) air can also give the user a general idea of what types of micro-organisms are airborne. Identification sticks and labels are provided to the user for each agar plate. In 1 to 5 days after placement of petri dish, the user can upload data to the Al platforms mobile phone app or desktop app. These applications will enable the user to upload pictures from a mobile phone or desktop computer for evaluation of data. After evaluation, the AI platform offers guidance on what possible steps are next or if any are required. The AI platform will provide elimination and micro-organism management solutions if required. Depending on the data, an AI platform may be required to be setup to gather more accurate data.

Another choice that the user has is the static electricity applied to microscope glass slides “slides” or “glass plate” application. The user will be provided a static electricity glass plate test kit. Basic components of the kit are standard with other options such as a blacklight.

Glass testing slides will be mailed, hand delivered or federal expressed if desired by the user. A box that separates 70 glass is shipped with two 6×6 inches sheets of wool and fur (user allergies are considered) In lieu of wool and fur, a sheet of plastic can be substituted. Instructions to the user have the user rubbing the glass slides on both sides with a sheet to create static electricity on the slide. The user will place the glass slides around the indoor structure indiscriminately while some slides are placed on countertops and sinks. The user should wait at least 24 hours for the slides to capture micro-organisms

For each 1,000 square feet, the application suggests 35 glass slides. After retrieving the slides for evaluation, the 35 slides must be covered by another slide for easy transporting to our AI platform for evaluation. Users can then choose to have the slides shipped to our facilities for evaluation or choose to have our artificial intelligence application platform set up in their facilities for accurate identification. The data evaluated is accurate and immediate. The artificial intelligence application can be set up in their homes or facilities for immediate evaluation.

“The Drone and Robot Test” for surface and airborne micro-organisms.

The second choice the user has is the scanning of the indoor facility by a drone and robot. If the facility is larger than 5,000 cubic feet and depending the amount of clutter, more than one drone and robot may be needed. The user can choose to utilize one drone and one robot in a large space but the duration of the process may be days instead of hours. The cost efficiency is available but timing may be unknown. There are instances when microbes are not present, but rare. This application can use basic environmental sensors such as humidity, air quality, gas and chemical sensors. The list of sensors that the user can choose from are listed at the end of the document.

This information will give the user a basic understanding of the level of pollutants that lurk in the structure as well as other basic data. This second choice offers another choice of a blacklight. The use of a blacklight (FIG. 17) on both the drone and robot can give the user an understanding of the level of feces and urine in the structure. This application is used to detect the possibility of the norovirus.

An electron microscope and sometimes a light microscope must be placed inside the structure for viewing of viruses captured on slides such as the norovirus. Or the slides can be forwarded to the AI platform for evaluation.

Initialization of the AI platform application.

The setup

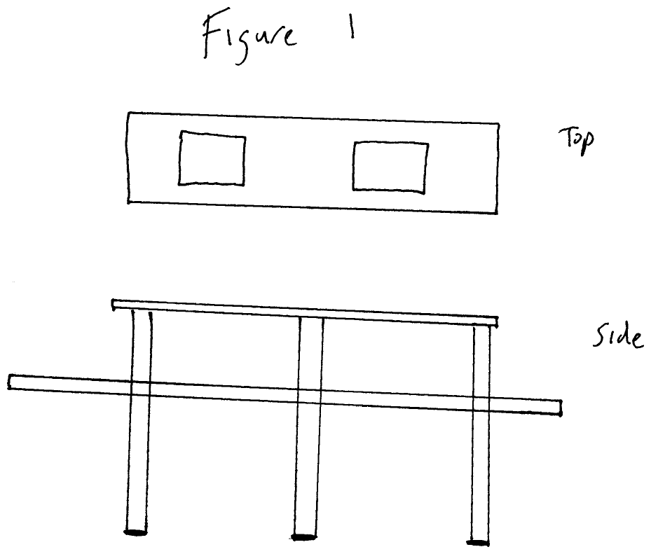

The transportation of the application is quite simple. A single platform is transported in three containers ranging from 40 pounds each to 135 pounds each. Container 1 contains a drone, a robot, and 2 computer screens. Container 2 contains the light microscope or an electron microscope, glass slides and Tram Cats. Container 3 contains the AI conveyor belt and the fold away table (some assembly is required), desktop computer, all other ancillary items such as batteries, light weight extension cords, cables, keyboard and mouse. FIG. 1 is of the double layered table.

The platform is that of a double leveled table with 6 legs. The double layered table is needed for the light microscope to where an AI conveyor belt with rectangular holes in the middle of the belt holds the glass slides for viewing by the microscope while the upper level of the table holds the light microscope, and a screen connected to the microscope and another screen for the desktop. At times, only one screen may be needed for conservation of space. Each step is numbered and color coded for quick assembly. FIG. 2 is of the Microscope. Can either be Electron or Light microscope.

While the platform is being setup, the drones and robots are programmed to can start evaluation. Once the AI platform is plugged in and goes online, the drone and robot will start to transfer the data collected so far.

Commencement of AI Platform Application. The start up

At the time of the drone in the air, the robot scanning and the power switch has been turned on, the AI platform takes over. The operation begins with scanning using lasers and environmental sensors of the indoor structure by the drones and robots while the system is being set up. More cost-efficient applications use one drone and one robot where the scanning will take more time. This information determines the humidity for static electricity applications and also maps out where the microscope slides (slides) can be placed. The drones and robots map out shelving, picture frames, cabinet tops and every cubic meter of the space for the machine learning applications to determine where the slides should be placed. The constant is that slides will always be placed on kitchen counters, restroom sinks, directly outside restrooms, on top of refrigerators and in vents.

The run time of the drones is 8 to 12 minutes utilizing batteries. The run time for robots on batteries is 12 to 21 minutes. The robot may use electrical outlets for power and manage the electrical cord by the AI platform. If there is a high degree of clutter in the space, much more time will be needed for the operation to start and complete. The machine learning application is learning how to plug and unplug from various electrical outlets. At times, a small additional drone and robot will be needed to manage the electrical cord if one is used. When the platform is operational, the gathered data from the runs from the drones and robots will be downloaded to the platform utilizing the docking station for drones and robots (FIG. 14). FIG. 9 is a Docking station. A 1 terabyte SanDisk “SD” card (secure digital memory card) stores the data and that data is easily transferred to the AI platform by two ways, removing the SD card physically or the more expensive way of docking the drone or robot to a male female connection on the AI platform. The platform is also a charging station for the batteries on the drones and robots. The physical removing of the SD card is available since some users regard the data on the card as personal.

Micro-Organism Attraction to Static Electricity

Before the placement of slides, the slides need to be charged by creating static electricity. Materials that tend to lose electrons (gain a positive charge) are glass, aluminum, nylon, wool and rabbit fur while materials that tend to attract electrons (gain a negative charge) is hard rubber, metals, polyester, styrofoam (styrene), saran wrap and vinyl. Metals tend to lose electrons while non-metals tend to gain electrons. The application uses positive and negative charges on slides made of glass, metals, silicon and plexiglass.

There are 3 manual ways in which the user creates static electricity: rubbing by hand the glass slides with another material such as a silk cloth or fur gloves. Rubbing together two sides of a plastic apron and then rubbing the glass. Unrolling saran wrap and then touching the glass. These are cheap options.

Placement of the slides by a user may not have the best outcome since some of the static electricity may be lost during placement. This is the last choice of manual involvement.

Creating Static Electricity with Tram Cats

When the application calls for Tram Cats, drones and robots will create the static electricity while performing initial scans. This particular application, saves time, money and creates a strong static electric field. Tram Cat containers are light, made of plastic and are a little bigger than the glass slides. Some have grills at both ends (or grill on 2 sides) so air can pass through them. The extra space is needed for slight movement between the two (plate and fabric) to create the static electricity. Select sides of the container are covered in fur. When using drones to create static electricity, prop wash directly over or under the Tram Cat provides vibration to create static electricity. Depending on the size of the drone, the vibration from the props and motors will also create static electricity. When using robots, knobby tires create vibration when the robot is in motion which in turn creates static electricity. When unpacked from the box, Tram Cat (FIG. 15) containers already have glass slides (FIG. 16) within them.

Number of loading and unloading of Tram Cats

The Tram Cats unload and load a total of 3 times each run. Starting with a Tram Cat just unpacked from the box, they need to have static electricity applied. They are unloaded to an indoor structure for obtaining micro-organisms (with glass plate). Then they (drone and or robot) during the trip from the initial startup to the placement site, they are charged with static electricity. Run 1 completed. Then, they are retrieved hopefully with micro-organisms. Some slides will not have any micro-organisms and must be recycled to restart the process. The Tram Cat (with glass plate and static electricity and micro-organism) are unloaded physically to the AI Platform AI conveyor belt for evaluation under the microscope (upload of glass plate) RUN 2. Then they are loaded back up after cleaning (with glass plate) for another run and to gain the static electricity on the ride. Run 3. Every 12 runs, the Tram Cat containers are retired and replaced with clean ones.

The processes of loading and unloading of Tram Cats

The Tram Cats are placed in the drones and robots in different manners. They also use 3 different ways of loading and unloading of Tram Cats.

Loading of Tram Cats via Robots

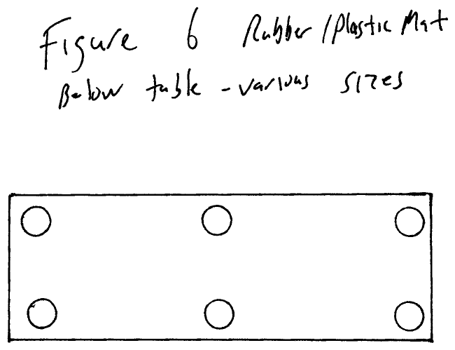

All robots are equipped with a mechanical arm. Some mechanical arms are magnetized and turn magnet fields on and off to hold the Tram Cat while the cheaper form of robot uses a mechanical arm to place the Tram Cat in and out of a basket affixed to the robot. FIG. 5 is of a mechanical arm on a robot in a docking station or a stand-alone mechanical arm. The mechanical arms are operated by a Command Stick “Joystick” FIG. 8 or by the Artificial Intelligence platform.

Unloading slides via Robots

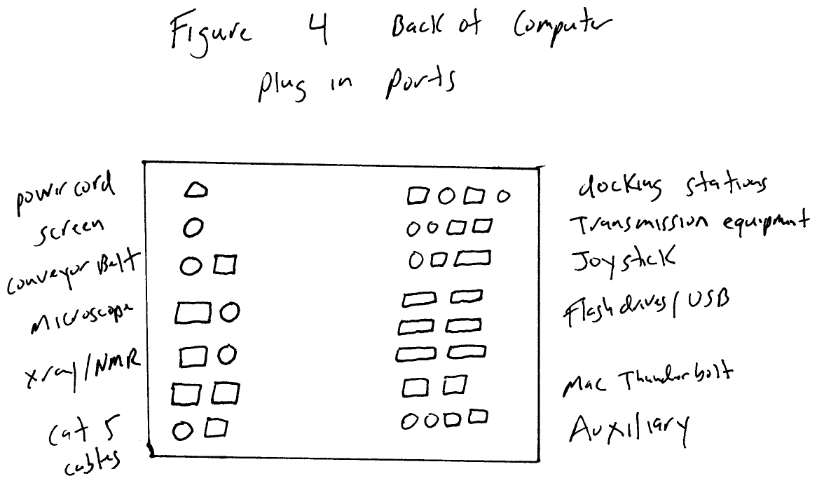

With the robot mechanical arm, removing the plate from the Tram Cat is quick and simple. The Tram Cat does not have to be removed from the basket of the robot, but can be removed. The robot can simply pull the plate out and place it on the AI conveyor belt. FIG. 3 on the lower level of the belt for evaluation. FIG. 16 is a plate.

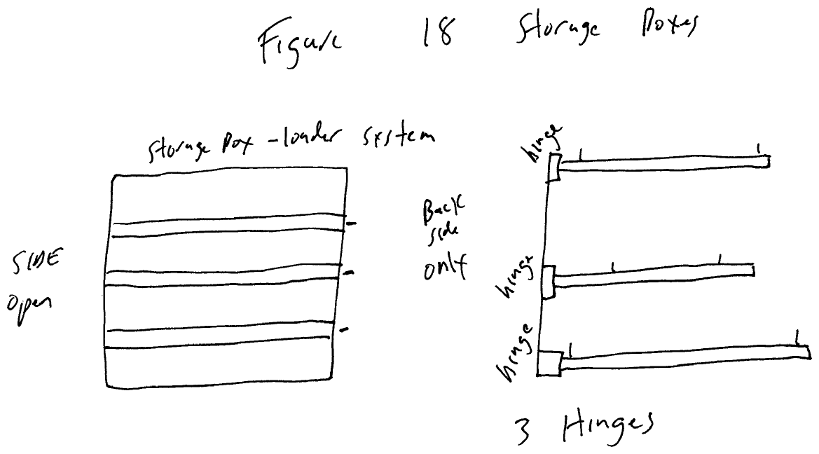

If the user prefers to maintain the sample of a certain finding, it will not be cleaned and pulled from the belt by the AI platform for further evaluation. The robot places the slide in a box to be stored at the end of the day. The AI Application uses a similar design to the Prior Scientific PLW20 Well Plate design. The slides are stored under the right side of table in the front and only the storage boxes are used. The robot hands off the slide to the loader system which swings out like a door. FIG. 18 is a storage box loader system. If the plate is not flagged by the AI platform, at the end of the belt the plate is turned upside down from the belt and dropped on a mat underneath the table. Slide may be stored in a refrigerator.

Cleaning the plate for reuse- degerming.

At the end of the belt after evaluation, the plate must be cleaned for reuse. The application uses an inexpensive way to rid the plate of micro-organisms. The plate is dipped in 91% Isopropyl alcohol. The mechanical arm of the robot will take the plate from the mat underneath the table and place it through a special Tram Cat container that is attached to the front left leg of the table. The Tram Cat container is closed and is water tight on all ends except for the top. Two cotton rolling pins at each side of the top of the open Tram Cat container will remove excess alcohol from the plate upon the robot removing the plate from the container. FIG. 10 is the washing station.

Placing the plate back in the Tram Cat -Robot

Immediately after the removal from the alcohol bath, the plate is ready to be deployed again. The robot places the plate back in the Tram Cat and the robot is off to deploy the plate for obtaining more micro-organisms for testing.

Placement of Tram Cats via Drone

The drone needs some help from the robot. The drone has a different Tram Cat placement technique. The drones must use magnets to hold the Tram Cat in place and release the magnetic field when the Tram Cat needs to placed. Since the outside of the Tram Cat Container is made of plastic, metal rails are affixed to the top and bottom which do not contact the inside of the Tram Cat. Magnets on the landing gear of the drones can pick up Tram Cats and drop off the Tram Cats with ease. Diagram 13 is a Drone landing gear with magnetic bars. With a flick of a switch, the magnetic field can be turned on or off (magnetic base).

Uploading of slides via Drone

The slides are retrieved by the drone in the same manner as the placement of the Tram Cat. The magnets on the landing gear are used to pick up the Tram Cat and deliver it to the floor of the platform where the robot will unload the slides as described in “Unloading slides via Robots”

The AI Conveyor Belt

After a power source is supplied and the platform is set up and operational, the automated application is managed by an artificial Intelligence platform. After the drone and robot start their initial run to obtain micro-organisms for evaluation (first phase), the accurate identification of micro-organisms (second phase) is the most important part of the AI platform. This is where Artificial Intelligence manages and machine learning offers choices.

How the AI platform of algorithms managing the conveyor belt “AI Conveyor Belt” operates using artificial intelligence and machine learning algorithms.

The most important aspect of this application is the ability to obtain an untainted sample micro-organism and to learn from the evaluations (data) of micro-organisms. The AI conveyor belt offers 4 separate applications.

Exact placement of slides with micro-organisms for accurate identity,

The ability to recognize trends, behaviors and the transport of micro-organisms for accurate “belt” positioning, evaluation and handling as to not taint the sample,

the ability to correct mistakes in the identification application And finally, belt management for storage of the sample for later research.

Exact placement of slides with micro-organisms for accurate identity

Working together for positioning and placement algorithms. Using Detailed GPS to position the plate for accurate identity of the plate combined with robots, mechanical arms and machine learning algorithms. The GPS “Global Positioning Software” utilizes a 3-dimensional software algorithm. The fourth calculation is of the plate itself. If the plate is not the right size, positioned in the wrong way (the issue is labeled and learned by machine learning algorithms and corrected by other “belt correction” algorithms), the 3-d software algorithm uses hi-def cameras and lasers on all 4 sides of the microscope with one 7 to 12 degrees off the top of the microscope being managed by the AI Platform to incorporate many steps to correct the issue.

The AI Conveyor Belt

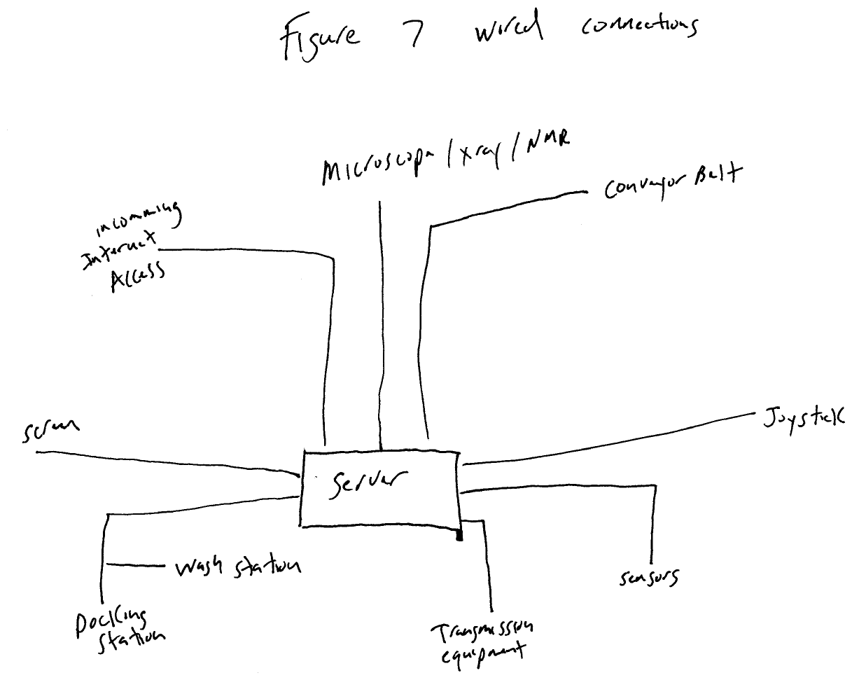

As in FIG. 3, the diagram shows the various components. In the middle of the belt, there is a strip of indents for the slides to lay exactly in that place without much movement. An example is that of a sim card being laid into a tray of a mobile phone that fits snugly. Slides may be turned over or around by the mechanical arm for another view of the micro-organism. Each AI conveyor belt has an average of 22 concave slots to hold the slides. The belt tray is see through where the bottom (beneath the microscope stage) of the microscope (condenser) has a direct line to the lens of the microscope and has nothing in its view other than the glass plate. Concave bottoms hold the slides from dropping through the belts. The belt can be sped up of slowed down by the AI platform. The slides are placed on the belt with a robot arm similar to the ones used in the Nikon BioPipeline Live or the Prior Scientific PLW20 Plate handling mechanical arms. The slides are removed by the belt dropping the slides when the belt arrives at section D FIG. 3 where the robots mechanical arm retrieves the plate from the rubber mat FIG. 6.

Sample Sequestering

If the Al platform evaluates something that is unknown to the system, that plate is tagged and removed from the rotation, not cleaned and stored for continued evaluation.

Identity markings from the AI platform.

The AI platform marks a plate by using a permanent magic marker to make a line/number by the mechanical arm. Each slide can have many different color lines. A plate that was marked as unknown on Apr. 2, 2021 at 4:00 pm will have a first line relate to level of threat (code red-unknown) second line is for the month (yellow line-April), third line day top half blue bottom half green (2ond day of Aril) fourth line (number 1 for year) fifth line time (number 0400).

The unforeseen

While Artificial Intelligence gives the appearance of a perfect application, there will be mistakes. The AI application works with a set of instructions. If there are any unforeseen occurrences, the AI Platform is in a constant stage of backups with redundant drives (RAID system) and the Al platform is designed to shut itself down when it encounters any unforeseen circumstances. The AI platform comes equipped with a identical backup system where the AI platform shuts itself down and waits for human intervention. The corporate option is to have an identical AI platform standing by (both hardware and software) that replaces the first platform.

Supporting Data Slides

Grainer White Glass Microscope slides 75×25 mm. The microscope can be a light microscope but for more accurate identity, an electron microscope can be used for micro-organisms up to 10 to the minus 8 power. For other applications, X-ray machines may be needed for micro-organisms that require 10 to the minus 10 power to identify.

Most of the times, the slides are placed on surfaces indoors. At times, the slides can be affixed to walls and ceilings using simple hooks attached to the slides, by velcro double sided tape. At some occasions, magnets may be used. The most inexpensive option of glass slides is to affix a plastic clip (all made of plastic) at one end of the glass plate.

The sizes of slides fit under a microscope or x-ray machine and range in size by the following: Meiji Glass clear slides for static electricity. 94.5 mm in diameter, Silicone Black White Microscope Stage plate 3/3/4 inch in diameter, New York Microscope company Glass stage plate 100 mm, Grace Bio-labs Culturewell Silicone Sheet Material ranging in size, Sheets of plexiglass ranging in various sizes and sheets of silicon ranging in size. The silicon may be laid over other materials. Slides may also have a Concave Depression Cavity.

Other Stationary Components

Laptops, tablets, mobile phones, chatbots, server farms, numerous connected server farms capable of exascale calculations, satellite transmission equipment (FIG. 11), solar panels for power, wind turbines, water mills for power generation, electrostatic generators, grounding machines, anti-static brushes, static eliminator bars, x-ray machines, electron microscopes and alcohol bathing stations.

Other Mobile Components

Manned and unmanned large (multi-ton) roaming vehicles on land, air and on (in) water, nanosized roaming vehicles on land, air and on (in) water, helicopters, robots that can also fly drones that can also move on the ground and or through floating balloons (filled with helium or hydrogen).

Other Tram Cat components Tram Cats can be made of plastic, metal, wood, rubber and cardboard.

Other sensors

Thermal image IR sensors, Thermal temperature and humidity sensors, infrared spot temperature sensor, stainless steel temperature, ultra-low temperature sensor, sound and noise level sensor, Air quality sensor, hydrogen gas sensor, Ozone gas sensor, airflow sensor, differential air pressure sensor, optical dust particle sensor, water leak sensor, fuel leak sensor, humidity sensor shock vibration sensor, smoke sensor, motion sensor, geophone sensor, microphone sensor, sound locator, air flow meter, curb feeler, wheel speed sensor, light sensor, mass airflow sensor, oxygen sensor, radar sensor, speed sensor, carbon dioxide sensor, carbon monoxide detector, electrochemical gas sensor, electronic nose, holographic sensor, hydrocarbon dewpoint analyzer, hydrogen sensor, infrared point sensor, ion selective electrode, nondispersive infrared sensor, nitrogen oxide sensor, nondispersive infrared sensor, oxygen sensor, smoke detector, magnetometer, metal detector, voltage detector, air pollution sensor, electrochemical gas sensor, gas detector, hygrometer, neutron detector, particle detector, altitude indicator, magnetic compass, flax sensor, impact sensor, lidar, linear encoder, odometer, shock detector, tilt sensor, tachometer, ultrasonic thickness gauge, electro optical sensor, flame detector, infrared sensor LED light sensor and fiber optic sensor wavefront sensor.

Laser producing equipment and laser sensors (FIG. 12)

1. An application using a platform where drones, robots, containers with glass microscope slides, static electricity applied to the glass microscope slides, conveyor belts, light microscopes or electron microscopes, wired and wireless data transmission equipment, desktop computers, servers, artificial intelligence and machine learning algorithms are used together to identify micro-organisms accurately.

2. An application using a platform managed by artificial intelligence and machine learning algorithms as a loading dock for a robot to load and unload glass microscope slides unto a conveyor belt running through a light or electron microscope to identify micro-organisms accurately.

3. An artificial intelligence application where a microscope slide, conveyor belt, light microscope or an electron microscope, artificial intelligence and machine learning algorithms identify micro-organisms accurately.

4. An application in claims 1 through 3 where glass microscope slides combined with static electricity attract micro-organisms from the air and from surfaces.

5. An application in claims 1 through 4 where two or more components are used simultaneously on a singular or connected platforms to identify micro-organisms accurately.

6. An application in claims 1 through 4 where the accurate identification of a micro-organism is obtained and the behaviors of the micro-organism are learned.

7. An application in claims 1 through 4 where components of the application can be completed manually.

8. An application in claims 1 through 4 where forecasts can be made utilizing the data obtained from the platform.

9. An application in claims 1 through 4 where the container has one or more sides lined with a fabric.

10. An application in claims 1 through 4 where the container has one or more sides lined with fur or wool.

11. An application in claims 1 through 7, 9 and 10 where the container has more than one glass microscope slide inside.

12. An application in claims 1 through 7, 9, 10 and 11 where the container is vibrated to create static electricity on a glass microscope slide.

13. An application in claims 1 through 7, and claims 9 through 12 where the prop and prop wash from a drone creates the vibration to produce the static electricity on the microscope slide.

14. An application in claim 1 through 7, and claims 9 through 12 where the rotation of at least one knobby tire on a robot creates the vibration to produce the static electricity on the microscope slide.

15. An application in claims 1 through 7, and claims 9 through 14 where the slides can be made of something other than glass.

16. An application in claims 1 through 3 where the light microscope or electron microscope is replaced with an x-ray machine or a Nuclear Magnetic Resonance “NMR” machine to obtain data and images.

17. An application in claim 1 where a robot and drone work together with lasers, mirrors and reflectors to obtain a 3-dimensional view of an indoor structure for microscope slide placement.

18. An application in claims 1 through 7, and claims 9 through 17 where the size of the application can be of normal scale, nanotechnology sized or a combination of both.
